(19) United States
(12) Patent Application Publication
(10) Pub. No.: US 2018/0243217 A1
(43) Pub. Date: Aug. 30, 2018

(54) NANOPARTICLES COMPRISING A STABILIZED BORONIC ACID COMPOUND

(71) Applicant: LEON-NANODRUGS GMBH, Munich (DE)

(72) Inventors: Kathrin Ines MÜHLHÖLZL-ODÖRFER, Munich (DE); Elke HORSTKOTTE, Munich (DE); Sabine WEITENSTEINER, Munich (DE); Isabel SOLE FONT, Munich (ES); Akif Emre TÜRELI, Saarlouis (TR)

(21) Appl. No.: 15/556,235

(22) PCT Filed: Mar. 11, 2016
(86) PCT No.: PCT/EP2016/055271

§ 371 Date: Sep. 6, 2017

(30) Foreign Application Priority Data
Mar. 17, 2015 (DE) 10 2015 103 873.0

(51) Int. Cl. A61K 9/14; A61K 38/05; A61K 9/00

(52) U.S. Cl. CPC A61K 9/145; A61K 9/0019; A61K 38/05

(57) ABSTRACT

The present invention provides nanoparticles comprising at least one boronic acid compound and at least one stabilizing agent for the at least one boronic acid compound and/or a reaction product of the at least one boronic acid compound and the at least one stabilizing agent, whereby the nanoparticles have a particle size of about 10 to about 1000 nm. The present invention also provides a pharmaceutical composition comprising these nanoparticles and a method for the preparation of these nanoparticles and the respective pharmaceutical compositions. In addition, the present invention provides nanoparticles and pharmaceutical compositions for the treatment of several disorders, especially multiple myeloma, preferably by parenteral administration.

The present invention is directed to nanoparticles comprising at least one boronic acid compound and at least one stabilizing agent for the at least one boronic acid compound and/or a reaction product of the at least one boronic acid compound and the at least one stabilizing agent. The present invention is also directed at a pharmaceutical composition comprising these nanoparticles. Further, the present invention is directed at these nanoparticles and pharmaceutical compositions, respectively, for parenteral administration, especially for subcutaneous administration and especially for the use in the treatment of various disorders, for example of multiple myeloma or mantle cell lymphoma. Furthermore, the present invention relates to a method for the preparation of the nanoparticles and the pharmaceutical composition, respectively. Finally, the present invention relates to the nanoparticles or the pharmaceutical composition, especially a suspension, obtainable by this method.

Boronic acid and ester compounds display a variety of pharmaceutically useful biological activities. U.S. Pat. No. 4,499,082 A discloses that peptide boronic acids are inhibitors of certain proteolytic enzymes. U.S. Pat. No. 5,187,157 A, U.S. Pat. No. 5,242,904 A and U.S. Pat. No. 5,250,720 A describe a class of peptide boronic acids that inhibit trypsin-like proteases. U.S. Pat. No. 5,169,841 A discloses N-terminally modified peptide boronic acid that inhibits the action of renin. U.S. Pat. No. 5,106,948 A discloses that certain tripeptide boronic acid compounds inhibit the growth of cancer cells.

U.S. Pat. No. 5,780,454 A, U.S. Pat. No. 6,066,730 A, U.S. Pat. No. 6,083,903 and U.S. Pat. No. 6,297,217 B1 describe peptide boronic ester and acid compounds useful as proteasome inhibitors. These prior art documents also describe the use of boronic ester and acid compounds to reduce the rate of muscle protein degradation, to reduce the activity of NF-κB in a cell, to reduce the rate of degradation of the p53 protein in a cell, to inhibit cyclin degradation in a cell, to inhibit the growth of a cancer cell, to inhibit antigen presentation in a cell, to inhibit NF-κB dependent cell adhesion and to inhibit HIV replication. WO 98/35691 A1 teaches that proteasome inhibitors, including boronic acid compounds, are useful for treating infarcts such as those that occur during stroke or myocardial infarction. WO 99/15183 A1 teaches that proteasome inhibitors are useful for treating inflammatory and autoimmune diseases. U.S. Pat. No. 6,958,319 B2 provides stable compounds prepared from boronic acid and lyophilized compounds thereof.

Unfortunately, alkylboronic acids are relatively difficult to obtain in analytically pure form. Snyder et al., J. Am. Chem. Soc., 3611 (1958), teaches that alkylboronic acid compounds readily form boroxines (anhydrides) under dehydrating conditions. Also, alkylboronic acids and their boroxines are often air-sensitive. Korcek et al., J. Chem. Soc., Perkin Trans. 2 242 (1972), teaches that butylboronic acid is readily oxidized by air to generate 1-butanol and boric acid. These difficulties limit the pharmaceutical utility of boronic acid compounds, complicating the characterization of pharmaceutical agents comprising boronic acid compounds and limiting their shelf life.

It is also well known in the art that specific boronic acid compounds, especially bortezomib, can be used in the treatment of patients suffering from multiple myeloma. Bortezomib is commercially available under the trade name VELCADE. It is offered as a lyophilized powder in form of the mannitol ester of boronic acid and has to be dissolved prior to use. Concentrations for parenteral administration are currently 1 mg/ml for i.v. and 2.5 mg/ml for s.c. application.

There is a need in the art for improved boronic acid compounds. Ideally, such compounds and their formulations, respectively would be conveniently prepared, most preferable as ready-to-use formulation without need of resuspension or dilution steps, would exhibit enhanced stability and longer shelf life as compared to the free boronic acid compound, and would readily liberate the active boronic acid compound when administered to a subject in need of boronic acid therapy. There is further a need for boronic acid compounds, especially for bortezomib, to be available as ready-to-use drugs, because presently boronic acid compounds, especially bortezomib, have to be dissolved prior to use, because they are not stable in aqueous solution for an appropriate period of time.

The present invention addresses these needs.

In a first aspect, the present invention is directed at nanoparticles comprising at least one boronic acid compound and at least one stabilizing agent for the at least one boronic acid compound and/or a reaction product of the at least one boronic acid compound and the at least one stabilizing agent, whereby the nanoparticles have a particle size of about 10 to about 1000 nm.

As employed herein, the term “boronic acid compound” refers to any chemical compound comprising a —B(OH), moiety. Snyder et al., J. Am. Chem. Soc. 3611 (1958), teaches that alkyl boronic acid compounds readily form oligomeric anhydrides by dehydration of the boronic acid moiety. Thus, unless otherwise apparent from the context, the term “boronic acid compound” is expressly intended to encompass free boronic acids, oligomeric anhydrides, including, but not limited to, dimers, trimers, and tetramers, and mixtures thereof.

The stabilizing agent has the function to stabilize the nanoparticles according to the present invention, especially in a pharmaceutical composition comprising the nanoparticles according to the present invention.

In a preferred embodiment, the at least one stabilizing agent is absorbed on the surface of the boronic acid compound which in turn improves the stability of the nanoparticles.

In another aspect, the nanoparticles comprise a reaction product of the at least one boronic acid compound and the at least one stabilizing agent. The reaction product preferably is formed by covalent bonding between the boronic acid compound and the stabilizing agent. In a preferred embodiment, the reaction product of the at least one boronic acid compound and the at least one stabilizing agent is a boronate ester.

It is also possible that the nanoparticles according to the present invention comprise both nanoparticles comprising at least one boronic acid compound and at least one stabilizing agent, the latter preferably absorbed on the surface of the boronic acid compound, and the reaction product of the at least one boronic acid compound and the at least one stabilizing agent.

The nanoparticles of the present invention are defined be their size of about 10 to about 1000 nm, which is based on the light intensity and measured as described hereinafter. More preferably, the nanoparticles of the present invention have a particle size of 100 to about 1000 nm, thus falling within the category of ‘fine’ nanoparticles according to standard definitions. Their size is defined as their diameter determined by a suitable process, e.g. using Dynamic Light Scattering (DLS) (e.g. using a Malvern Zetasizer ZS90 from Malvern Instruments Ltd.). DLS measures Brownian motion and relates this to the size of the particles. Brownian motion is the random movement of particles due to the bombardment by the solvent molecules that surround them. The larger the particle or molecule, the slower the Brownian motion will be. Smaller particles are “kicked” further by the solvent molecules and move more rapidly. An accurately known temperature is necessary for DLS because knowledge of the viscosity is required (because the viscosity of a liquid is related to its temperature). In the present measurement a temperature of 25° C. is used. This temperature is kept constant during the measurement. The velocity of the Brownian motion is defined by the translational diffusion coefficient (D). The size of a particle is calculated from the translational diffusion coefficient by using the Stokes-Einstein equation;

$$d ( H ) = \frac{kT}{3 π η D}$$

Where d(H) is the hydrodynamic diameter, D is the translational diffusion coefficient, k is the Boltzmann's constant, T is the absolute temperature, and η is the viscosity. The diameter that is obtained by the Stokes-Einstein equation is the diameter of a sphere that has the same translational diffusion coefficient as the particle. The particle translational diffusion coefficient will depend not only on the size of the particle “core”, but also on any surface structure that will affect the diffusion speed, as well as the concentration and type of ions in the medium. Malvern Zetasizer series mesure the speed at which the particles diffuse due to Brownian motion by determining the rate at which the intensity of scattered light fluctuates when detected using a suitable optical arrangement. In the Zetasizer Nano ZS90 series, the detector position is 90°.

The z-average diameter, together with the polydispersity index (PDI), are calculated from the cumulants analysis of the DLS measured intensity autocorrelation function as defined in ISO22412:2008. PDI is a dimensionless estimate of the width of the particle size distribution, scaled from 0 to 1.

According to Malvern Instruments, samples with PDI 0,4 are considered to be monodisperse.

In a preferred embodiment, the at least one boronic acid compound comprised in the nanoparticles according to the present invention has the following formula (I):

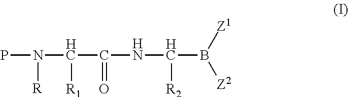

or a pharmaceutically acceptable salt thereof; wherein

P is R⁴—C(O)— or R⁴—SO₂—, where R⁴ is quinolinyl, pyrazinyl, pyridyl, quinoxalinyl, furyl, pyrrolyl, or N-morpholinyl;

R is hydrogen or alkyl;

R¹ and R² are independently selected from the group consisting of hydrogen, alkyl, cycloalkyl, aryl, heterocycle, and —CH₂—R⁵, where R⁵, in each instance, is one of aryl, aralkyl, alkaryl, cycloalkyl, or

—W—R⁶, where W is a chalcogen and R⁶ is alkyl;

where the ring portion of any of said aryl, aralkyl or alkaryl in R¹, R² and R⁵ can be optionally substituted by one or two substituents independently selected from the group consisting of C₁₋₆ alkyl, C₃₋₈ cycloalkyl, C₁₋₆alkyl(C₃₋₈)cycloalkyl, C₂₋₈ alkenyl, C₂₋₈ alkynyl, cyano, amino, C₁₋₆alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀aryl(C₁₋₆)alkyl, C₆₋₁₀ aryl(C₁₋₆)alkoxy, hydroxy, C₁₋₆ alkylthio, C₁₋₆alkylsulfinyl, C₁₋₆ alkylsulfonyl, C₆₋₁₀ arylthio, C₆₋₁₀ arylsulfinyl, C₆₋₁₀arylsulfonyl, C₆₋₁₀ aryl, C₁₋₆ alkyl(C₆₋₁₀)aryl, and halo(C₆₋₁₀)aryl; and Z1 and Z2 are both hydroxy.

In a very preferred embodiment, P is R⁴—C(O)— where R⁴ is pyrazinyl.

In a further very preferred embodiment, R is hydrogen.

In a further very preferred embodiment, R¹ is —CH₂—R⁵, where R⁵ is aryl and R² is alkyl.

In a very preferred embodiment, the at least one boronic acid compound having the above mentioned formula (I) is [(1R)-3-methyl-1-({(2S)-3-phenyl-2-[(pyrazin-2-ylcarbonyl) amino]propanoyl}amino)butyl]boronic acid. This compound is also known as bortezomib and has the following structure:

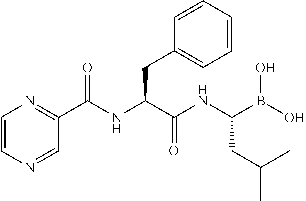 [SMILES: CC(C)C[C@H](NC(=O)[C@H](Cc1ccccc1)NC(=O)c1cnccn1)B(O)O]

In a further preferred embodiment, the reaction product of the at least one boronic acid compound and the at least one stabilizing agent has the following formula (II):

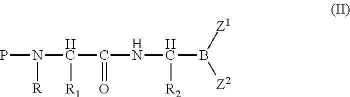

or a pharmaceutically acceptable salt thereof; wherein

P is R⁴—C(O)— or R⁴—SO₂—, where R⁴ is quinolinyl, pyrazinyl, pyridyl, quinoxalinyl, furyl, pyrrolyl, or N-morpholinyl;

R is hydrogen or alkyl;

R¹ and R² are independently selected from the group consisting of hydrogen, alkyl, cycloalkyl, aryl, heterocycle, and —CH₂—R⁵,

where R⁵, in each instance, is one of aryl, aralkyl, alkaryl, cycloalkyl,

or —W—R⁶, where W is a chalcogen and R⁶ is alkyl;

where the ring portion of any of said aryl, aralkyl or alkaryl in R¹, R² and R⁵can be optionally substituted by one or two substituents independently selected from the group consisting of C₁₋₆ alkyl C₃₋₈ cycloalkyl, C₁₋₆alkyl(C₃₋₈)cycloalkyl, C₂₋₈ alkenyl, C₂₋₈ alkynyl, cyano, amino, C₁₋₆alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀aryl(C₁₋₆)alkyl, C₆₋₁₀ aryl(C₁₋₆)alkoxy, hydroxy, C₁₋₆ alkylthio, C₁₋₆alkylsulfinyl, C₁₋₆ alkylsulfonyl, C₆₋₁₀ arylthio, C₆₋₁₀ arylsulfinyl, C₆₋₁₀arylsulfonyl, C₆₋₁₀ aryl, C₁₋₆ alkyl(C₆₋₁₀)aryl, and halo(C₆₋₁₀)aryl;

wherein Z¹ and Z² together form a moiety derived from the at least one stabilizing agent, wherein the atom attached to boron in each case is an oxygen atom.

In a very preferred embodiment, P is R⁴—CO(—), where R⁴ is pyrazinyl.

In a further very preferred embodiment, R is hydrogen.

In a further very preferred embodiment, R¹ is CH₂—R⁵, where R⁵ is aryl and R² is alkyl.

In a preferred embodiment, the at least one stabilizing agent is selected from the group comprising phosphatidylglycerol, vitamin E, vitamin E TPGS, deoxycholic acid, sodium deoxycholate, oleic acid, sodium oleate, phosphatidylcholine, preferably phosphatidylcholine from sojabeans (Lipoid S100) and/or polyethylene glycol, preferably PEG 200.

As employed herein, the term “phosphatidylglycerol” refers to any glycerol substituted at the 3-position by a phosphatidyl group.

In a particularily preferred embodiment, the reaction product of the at least one boronic acid compound and the at least one stabilizing agent is

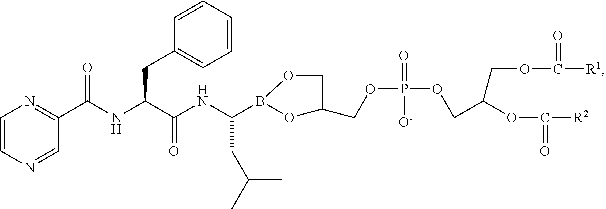

wherein R¹ and R² are fatty acid side chains.

Preferably R¹ and R² are alkyl chains, preferably C₆- to C₂₂-alkyl chains with a maximum number of unsaturations of six. R¹ and R² can be different.

As stated above, the nanoparticles according to the present invention have a particle size of about 10 to about 1000 nm. Preferably the nanoparticles according to the present invention have a particle size of about 70 nm to about 1000 nm, more preferably of about 70 nm to about 500 nm and most preferably of about 100 nm to about 200 nm. A particle size of about 100 nm to about 200 nm has the advantage that those particle sizes are regarded as fine particles in contrast to particles below 100 nm, but however, display a high surface area. Exponential increase in the surface area for particles in the nanometer range leads to a significant decrease in dissolution time as well as to an increase in the saturation solubility.

In a preferred embodiment, the nanoparticles according to the present invention have a polydispersity index of ≤ about 0.5, preferably of ≤ about 0.25 and more preferably of about ≤0.2.

The polydispersity index (PDI) is a parameter to define the particle size distribution of the nanoparticles obtained from dynamic light scattering (DSL) measurements. As mentioned above, the PDI might be measured using a Malvern Zetasizer according to the manufacturer's instructions. The smaller the PDI value is, the lower the degree of particle size distribution. Generally, polydispersity index PDI is used as degree of particle size distribution. Thus, particles/particle suspensions may be generally divided into monodisperse and polydisperse entities. For monodisperse, e.g. homogenous suspensions/particles, a tight particle size distribution is given. For polydisperse suspensions/particles, particle sizes vary considerably.

Particle size, as well as the PDI are important factors affecting the dissolution rate of particular substances, e.g. pharmaceutical active ingredients. Thus, comparison of dissolution of two nanoparticular populations of one active pharmaceutical ingredient with comparable mean particle sizes but significantly differing PDI might result in significant change in dissolution behavior of those nanoparticles, with slower dissolution for the nanoparticles with higher PDI and faster dissolution for the nanoparticles with lower PDI. Thus, PDI might affect, beside particle size, the quality of nanoparticles.

In a further aspect, the present invention is directed at a pharmaceutical composition comprising the nanoparticles of the present invention.

In a preferred embodiment, the nanoparticles are stable in the pharmaceutical composition. By stable is meant that the nanoparticles contained in the pharmaceutical composition have sufficient stability in order to have utility as a pharmaceutical agent. Preferably, the pharmaceutical composition has sufficient stability to allow storage at a convenient temperature, preferably between about 0° C. and about 40° C., for a reasonable period of time, preferably longer than one month, more preferably longer than three months, even more preferably longer than six months, and most preferably longer than one year. It is believed that the stabilizing of the nanoparticles in the pharmaceutical compositions occurs mainly in view of sterical reasons.

The pharmaceutical composition according to the present invention may be any dosage form commonly used for pharmaceutical administration, like for example, solids, as for example tablets and capsules, liquids, suspensions, creams, gels, ointments, emulsions, depots, etc. Preferably the pharmaceutical composition is a suspension, more preferably an aqueous suspension, which has the advantage that it can be readily administered to a patient without need of prior dilution.

The nanoparticles and the pharmaceutical composition comprising the nanoparticles, respectively exhibit an enhanced stability and a longer shelf life compared to the free boronic acid compound.

The nanoparticles according to the present invention protect the boronic acid compound, that is the active ingredient, from chemical degradation, especially in an aqueous medium, and the storage stability is improved. In addition, the nanoparticles of the present invention lead to the possibility of realizing a ready-to-use dosage form, especially a ready-to-use aqueous colloidal suspension. One major advantage of the pharmaceutical composition, especially in form of an aqueous suspension, is that a dissolution prior to administration is not necessary, which is a disadvantage of the product “VELCADE”.

The pharmaceutical composition preferably comprises additional pharmaceutically acceptable excipients.

In addition, the pharmaceutical composition of the present invention can comprise further additional active ingredients, like for example antiproliferative, cytotoxic or immunosuppressive agents.

In a further aspect the present invention is directed at the above mentioned nanoparticles and the above mentioned pharmaceutical composition, respectively for oral, pulmonary and nasal, topical and parenteral administration, preferably parenteral administration, to a mammalian subject, preferably a human. In all cases the nanoparticles dissolve almost immediately upon systemic administration and readily release the boronic acid compound. Especially, pharmacokinetics will preferably be comparable to the marketed product Velcade in means of area under the curve AUCₗₐₛₜ (given in ng*h/mL±SD), but might differ in means of cmax and tmax. Pharmacokinetic parameters of Velcade in patients with relapsed multiple myeloma comparing subcutaneous (155±56.8) vs. intravenous application (151±42.9) are published by Moreau et al. (Moreau P et al. Subcutaneous versus intravenous administration of bortezomib in patients with relapsed multiple myeloma: a randomised, phase 3, non-inferiority study. Lancet Oncol. 2011; 12:431-40). Single dose as well as multiple dose PK data comparing subcutaneous (single dose: 92.1±17.8; multiple dose: 195±51.2) vs. intravenous (single dose: 104±99.0; multiple dose: 241±82.0) application are available to the public via the assessment report of the European Medicines Agency EMA and derive from Phase III study 26866138-MMY-3021, an open-label, randomized study in subjects with relapsed multiple myeloma after prior systemic therapy (http://www.ema.europa.eu/docs/en_GB/document_library/EPAR_-_Assessment_Report_-_Variation/human/000539/WC500133654.pdf).

In a further aspect, the above mentioned nanoparticles or the above mentioned pharmaceutical composition are suitable for use in the treatment of malignant hematological disorders, like for example multiple myeloma, colorectal cancer, lung cancer, pancreatic cancer, breast cancer, prostate cancer, ovarian cancer or non-hodgkin-lymphoma. Especially preferred is the use in the treatment of multiple myeloma and mantle cell lymphoma.

It is clear to the skilled artisan that the nanoparticles and the pharmaceutical composition comprising them, respectively are effective in the treatment of the above mentioned diseases, because it is known that the boronic acid compounds and their derivatives, respectively, especially bortezomib, readily liberate the active boronic acid compound when administered to a subject in need of boronic acid therapy.

In a further aspect the present invention is directed at a method for the preparation of nanoparticles or a pharmaceutical composition comprising nanoparticles, which method comprises the steps of

a) providing a fluid mixture of at least one boronic acid compound and at least one stabilizing agent in an organic solvent and a fluid non-solvent,

b) precipitating the nanoparticles by colliding the fluid mixture and the fluid non-solvent, and

c) optionally, evaporation of the organic solvent and the fluid non-solvent.

Accordingly, the at least one boronic acid compound and the at least one stabilizing agent are dissolved in an organic solvent which is miscible or immiscible with water.

The term “fluid mixture” as used herein denotes a mixture of the at least one boronic acid compound and the at least one stabilizing agent in a solvent. A solvent is any kind of fluid substance which is capable of dissolving the at least one boronic acid compound.

Although the term “fluid” as used in the present specification includes liquids, gases and plasmas according to standard definition, it usually means a substance which is liquid at room temperature (21° C.).

The term “non-solvent” according to the present invention describes any fluid substance which is capable of precipitating boronic acid containing nanoparticles by colliding a fluid stream of it with a fluid stream of the fluid mixture. Therefore, a “non-solvent” in the meaning of the present invention should not be interpreted narrowly, for example as a substance in which boronic acid compounds are insoluble.

In case the organic solvent is miscible with water, the organic solvent is preferably methanol, ethanol, t-butanol, acetone, dimethylsulfoxide DMSO, or mixtures thereof. In case the organic solvent is immiscible with water, the organic solvent is preferably ethyl acetate, methylene chloride or mixtures thereof.

The fluid non-solvent is preferably water. Thus, the precipitation is realized preferably against water.

With that method nanoparticles having a particle size of about 10 to about 1000 nm comprising at least one boronic acid compound and at least on stabilizing agent for the at least one boronic acid compound and/or a reaction product of the at least one boronic acid compound and the at least one stabilizing agent are obtained.

Preferably phosphatidylglycerol is used as stabilizing agent. In that case the glycerol moiety in the hydrophilic portion of the phosphatidylglycerol will be attached to the boron atom by the oxygen atoms of its hydroxyl groups, resulting in a boronate ester formation.

Preferably, the boron atom, the oxygen atoms attached to the boron atom, and the atoms connecting to oxygen atoms together form a 5-membered ring. Particles of sterically stabilized boronic acid by the hydrophobic moieties of the molecules will be formed.

It is also possible that the stabilizing agent is adsorbed on the surface of the boronic acid compound. This also leads to a sterically stabilized boronic acid compound in form of nanoparticles. Preferred stabilizing agents are phosphatidylglycerol, vitamin E, vitamin E TPGS, deoxycholic acid, sodium deoxycholate, oleic acid, sodium oleate, phosphatidylcholine, more preferably phosphatidylcholine from soybeans (Lipoid S100), and/or polyethylene glycol, more preferably PEG200.

It is also possible that more than one stabilizing agent is used.

In case it is desired that the nanoparticles are obtained as solid nanoparticles, the organic solvent and the fluid non-solvent are evaporated, preferably under vacuum. Of course, it is also possible to isolate the nanoparticles as a suspension comprising the nanoparticles, especially as an aqueous suspension.

Preferably, the volume ratio of the solvent and the non-solvent is between 1:1 and 1:10, more prefarably between 1:1 and 1:5, more preferably between 1:1 and 1:2.

The methods of the present invention thus preferably includes controlled solvent/non-solvent precipitation, where solvent and non-solvent streams collide as impinging jets with a high velocity of more than 1 m/sec, where the Reynold number is higher than 500. The velocity, in one embodiment, may be higher than 50 m/sec as well. It is noted that the above indicated velocity is the velocity of each of the colliding streams, i.e., both the fluid stream of the fluid mixture and the fluid stream of the non-solvent have this velocity.

The solvent and the non-solvent preferably are sprayed through nozzles usually smaller than about 1000 μm (for example smaller than about 500 μm or about 300 μm) with pressures of more than about 1 bar. Pressures of more than about 10 bars and even more than about 50 bar are suitable as well. The pressure may be regulated by pressure regulators.

The two streams collide in a reactor, where a very rapid mixing takes place. Mixing times usually are below about 1 millisecond, preferably below about 0.5 milliseconds, and even more preferably under about 0.1 millisecond. The flow rates of solvent and non-solvent streams may reach more than about 600 l/hour. Thus, the two impinging jets (or streams) collide in the reactor where precipitation takes place forming disc like structures depending on the reactor geometry.

The mixing time is adjusted as a derivative of the flow rate, the higher the flow rate, the lower the mixing time will be. The mixing is done in the molecular state. In the reactor, where the fluid streams collide, two plates are formed because of the parallel streams flowing against each other. Then, the diffusion process starts from solvent to non-solvent and at the end of this diffusion, the mixture is completed. This time period can be controlled with the flow rate and also the gas pressure. This kind of mixing is preferably obtained with a so called microjet reactor since its structure allows the collision of two streams in a free chamber under gas so that the particle size can be controlled.

The term “microjet reactor” includes all the geometries that are defined in WO 0061275 A2. The contents of this patent application is incorporated herein by reference. WO 0061275 A2 provides for a system for the initiation of chemical or physical processes including at least two liquid media to be injected by means of pumps, preferably high-pressure pumps, into a reactor chamber enclosed by a reactor housing and on to a shared collision point, each medium being injected through one nozzle. Through an opening in the reactor chamber, a gas, an evaporating liquid, a cooling liquid or a cooling gas, is introduced so as to maintain the gas atmosphere in the reactor interior, notably in the collision point of the liquid jets, and to cool the resulting products. The resulting products and excess gas are removed from the reactor housing via a further opening by positive pressure on the gas input side or negative pressure on the product and gas discharge side.

The nanoparticles formed as described above are then preferably further processed to the final pharmaceutical formulation. In case that the final pharmaceutical formulation is an aqueous nanosuspension for parenteral administration preferably first organic solvents have to be removed according to set authority limits. This can be realized by using a diafiltration or lyophilization process. pH and osmolarity can be readily adjusted during the diafiltration process, accordingly. If a solid dosage form is targeted, the whole nanoparticle suspension is preferably further processed by a drying process (e.g. wet granulation or fluid bed granulation, spray drying). The obtained powder can be further processed by common pharmaceutical processes.

Regarding specific embodiments of the at least one boronic acid compound used in the method according to the present invention, it is referred to the above embodiments discussed in connection with the product claims.

The nanoparticles thus can be designed to be used in a variety of different pharmaceutical compositions and formulations, such as oral delivery as tablets, capsules or suspensions, pulmonary and nasal delivery, topical delivery as emulsions, ointments and creams, and parenteral delivery as suspensions, microemulsions or as a depot. Most preferred is parenteral delivery.

In a further aspect the present invention is directed at the nanoparticles and the pharmaceutical composition, preferably the suspension, obtainable by the above mentioned process.

The present invention is now described in more detail by the following examples. However, it is noted that the examples are provided for illustrative purposes only, and should not be construed to limit the scope of the invention in any way.

### EXAMPLES

### Example 1

Bortezomib was dissolved in ethanol together with phosphatidylglcerol in EtOH. This solvent mixture was precipitated against water using microjet reactor revealing nanoparticle suspension with a particle size of 170 nm and a PDI of 0.15.

### Example 2

Ethanol was removed from the nanosuspension prepared above using diafiltration. Water was used as exchange medium. Five volume exchanges were conducted in a continuous mode.

### Example 3

Stability testing of formulation of Example 1 and 2 at refrigerated, room temperature as well as accelerated conditions (40° C.) showed stability of the aqueous nanosuspension over at least 6 months.

### Example 4

Bortezomib was dissolved in DMSO together with phosphatidylglycerol in DMSO. This solvent mixture was precipitated against water using microjet reactor revealing nanoparticle suspension with a particle size of 190 nm and a PDI of 0.20.

1. Nanoparticles comprising at least one boronic acid compound and at least one stabilizing agent for the at least one boronic acid compound and/or a reaction product of the at least one boronic acid compound and the at least one stabilizing agent, whereby the nanoparticles have a particle size of about 10 to about 1000 nm.

2. Nanoparticles according to claim 1, wherein the at least one boronic acid compound has the following formula (I): 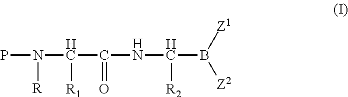
or a pharmaceutically acceptable salt thereof; wherein
P is R⁴—C(O)— or R⁴—SO₂—, where R⁴ is quinolinyl, pyrazinyl, pyridyl, quinoxalinyl, furyl, pyrrolyl, or N-morpholinyl;
R is hydrogen or alkyl;
R¹ and R² are independently selected from the group consisting of hydrogen, alkyl, cycloalkyl, aryl, heterocycle, and —CH₂—R⁵,
where R⁵, in each instance, is one of aryl, aralkyl, alkaryl, cycloalkyl,
or —W—R⁶, where W is a chalcogen and R⁶ is alkyl;
where the ring portion of any of said aryl, aralkyl or alkaryl in R¹, R² and R⁵ can be optionally substituted by one or two substituents independently selected from the group consisting of C₁₋₆ alkyl, C₃₋₈ cycloalkyl,
C₁₋₆alkyl(C₃₋₈)cycloalkyl, C₂₋₈ alkenyl, C₂₋₈ alkynyl, cyano, amino, C₁₋₆alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀aryl(C₁₋₆)alkyl, C₆₋₁₀ aryl(C₁₋₆)alkoxy, hydroxy, C₁₋₆ alkylthio,
C₁₋₆alkylsulfinyl, C₁₋₆ alkylsulfonyl, C₆₋₁₀ arylthio, C₆₋₁₀ arylsulfinyl, C₆₋₁₀arylsulfonyl, C₆₋₁₀ aryl, C₁₋₆ alkyl(C₆₋₁₀)aryl, and halo(C₆₋₁₀)aryl; and
Z¹ and Z² are both hydroxy.

3. Nanoparticles according to claim 2, wherein the compound of formula (I) is [(1R)-3-methyl-1-({(2S)-3-phenyl-2-[(pyrazin-2-ylcarbonyl) amino]propanoyl}amino)butyl]boronic acid.

4. Nanoparticles according to claim 1, wherein the reaction product of the at least one boronic acid compound and the at least one stabilizing agent has the following formula (II): 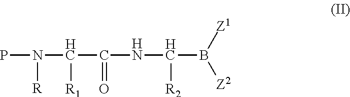
or a pharmaceutically acceptable salt thereof; wherein
P is R⁴—C(O)— or R⁴—SO₂—, where R⁴ is quinolinyl, pyrazinyl, pyridyl, quinoxalinyl, furyl, pyrrolyl, or N-morpholinyl;
R is hydrogen or alkyl;
R¹ and R² are independently selected from the group consisting of hydrogen, alkyl, cycloalkyl, aryl, heterocycle, and —CH₂—R⁵,
where R⁵, in each instance, is one of aryl, aralkyl, alkaryl, cycloalkyl, or —W—R⁶, where W is a chalcogen and R⁶ is alkyl;
where the ring portion of any of said aryl, aralkyl or alkaryl in R¹, R² and R⁵ can be optionally substituted by one or two substituents independently selected from the group consisting of C₁₋₆ alkyl C₃₋₈ cycloalkyl,
C₁₋₆alkyl(C₃₋₈)cycloalkyl, C₂₋₈ alkenyl, C₂₋₈ alkynyl, cyano, amino,
C₁₋₆alkylamino, di(C₁₋₆)alkylamino, benzylamino, dibenzylamino, nitro, carboxy, carbo(C₁₋₆)alkoxy, trifluoromethyl, halogen, C₁₋₆ alkoxy, C₆₋₁₀ aryl, C₆₋₁₀aryl(C₁₋₆)alkyl, C₆₋₁₀ aryl(C₁₋₆)alkoxy, hydroxy, C₁₋₆ alkylthio,
C₁₋₆alkylsulfinyl, C₁₋₆ alkylsulfonyl, C₆₋₁₀arylthio, C₆₋₁₀ arylsulfinyl, C₆₋₁₀arylsulfonyl, C₆₋₁₀ aryl, C₁₋₆ alkyl(C₆₋₁₀)aryl, and halo(C₆₋₁₀)aryl;
wherein Z¹ and Z² together form a moiety derived from the at least one stabilizing agent, wherein the atom attached to boron in each case is an oxygen atom.

5. Nanoparticles according to claim 1, wherein the at least one stabilizing agent is selected from the group comprising phosphatidylglycerol, vitamin E, vitamin E TPGS, deoxycholic acid, sodium deoxycholate, oleic acid, sodium oleate, phosphatidylcholine and/or polyethylene glycol.

6. Nanoparticles according to claim 1, wherein the at least one boronic acic compound is [(1R)-3-methyl-1-({(2S)-3-phenyl-2-[(pyrazin-2-ylcarbonyl)amino]propanoyl}amino)butyl]boronic acid and wherein the at least one stabilizing agent is phosphatidylglycerol and/or wherein the reaction product of the at least one boronic acid compound and the at least one stabilizing agent is 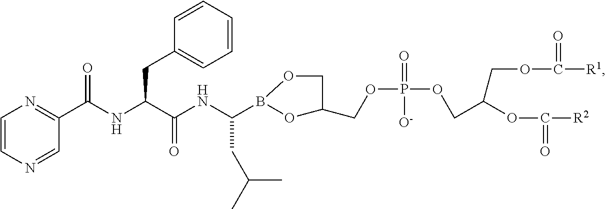
wherein R¹ and R² are fatty acid side chains.

7. Nanoparticles according to claim 1, wherein the nanoparticles have a particle size of about 70 nm to about 1000 nm.

8. Nanoparticles according to claim 1, wherein the nanoparticles have a polydispersity index of ≤about 0.5.

9. Pharmaceutical composition comprising the nanoparticles of claim 1.

10. Pharmaceutical composition of claim 9, wherein the pharmaceutical composition is an aqueous suspension.

11. Nanoparticles according to claim 1, for parenteral administration.

12. Nanoparticles according to claim 1 or pharmaceutical composition according to any one of claim 9 or 10 providing comparable area under the curve AUC levels as the marketed product Velcade, where AUCₗₐₛₜ is comparable or even-shows bioequivalence in means of 90% confidence interval to the Velcade AUC of
155±56.8 in ng*h/mL±SD after single subcutaneous administration in patients with relapsed multiple myeloma,
151±42.9 in ng*h/mL±SD after single intraveneous administration in patients with relapsed multiple myeloma,
92.1±17.8 in ng*h/mL±SD after single subcutaneous administration in patients with relapsed multiple myeloma after prior systemic therapy,
104±99.0 in ng*h/mL±SD after single intraveneous administration in patients with relapsed multiple myeloma after prior systemic therapy,
195±51.2 in ng*h/mL±SD after multiple subcutaneous administration in patients with relapsed multiple myeloma after prior systemic therapy, or
241±82.0 in ng*h/mL±SD after multiple intraveneous administration in patients with relapsed multiple myeloma after prior systemic therapy

13. Nanoparticles according to claim 1 for use in the treatment of malignant hematological disorders, like for example multiple myeloma, colorectal cancer, lung cancer, pancreatic cancer, breast cancer, prostate cancer, ovarian cancer or non-hodgkin-lymphoma.

14. Nanoparticles or pharmaceutical composition according to claim 11 for use in the treatment of multiple myeloma or mantle cell lymphoma.

15. Method for the preparation of the nanoparticles according to comprising the steps of
a) providing a fluid mixture of at least one boronic acid compound and at least one stabilizing agent in an organic solvent and a fluid non-solvent,
b) precipitating the nanoparticles by colliding the fluid mixture and the fluid non-solvent, and
c) optionally, evaporation of the organic solvent and the fluid non-solvent.

16. (canceled)

17. Nanoparticles according to claim 1, wherein the nanoparticles have a polydispersity index of ≤ about 0.25.

18. Nanoparticles according to claim 1, wherein the nanoparticles have a polydispersity index of ≤ about 0.2.

19. Nanoparticles according to claim 1 for subcutaneous or intravenous administration.

20. Nanoparticles according to claim 1, wherein the nanoparticles have a particle size of about 100 nm to about 200 nm.

21. Pharmaceutical composition of claim 9, wherein the pharmaceutical composition is a ready to use suspension without need of further dilution.
